(19) United States
(12) Patent Application Publication
(10) Pub. No.: US 2007/0275031 A1
(43) Pub. Date: Nov. 29, 2007

(54) Implantable polymeric device for sustained release of buprenorphine with minimal initial burst

(76) Inventors: Rajesh A. Patel, Redwood City, CA (US); Louis R. Bucalo, Miami, FL (US)

Correspondence Address: MORRISON & FOERSTER LLP, 755 PAGE MILL RD, PALO ALTO, CA, 94304-1018, US

(21) Appl. No.: 11/805,608

(22) Filed: May 23, 2007

Related U.S. Application Data
(60) Provisional application No. 60/802,999, filed on May 23, 2006.

(51) Int. Cl. A61K 31/282; A61F 13/00

(52) U.S. Cl. 424/422; 514/282

(57) ABSTRACT

The present invention provides compositions, methods, and kits for treatment of pain. The invention provides a biocompatible nonerodible polymeric device which releases buprenorphine continuously with generally linear release kinetics for extended periods of time, and exhibits minimal initial burst upon subcutaneous implantation. Buprenorphine is released through pores that open to the surface of the polymeric matrix in which it is encapsulated. The device may be administered subcutaneously to an individual in need of continuous treatment with buprenorphine. Devices of the invention are washed with ethanol for greater than 30 minutes prior to subcutaneous implantation or have release characteristics of a device that has been washed with ethanol for greater than 30 minutes, such as a low peak to steady state plasma level ratio.

### CROSS-REFERENCE TO RELATED APPLICATIONS

This application claims the benefit of U.S. Provisional Application No. 60/802,999, filed on May 23, 2006, which is incorporated by reference herein in its entirety.

### TECHNICAL FIELD

The invention provides a nonbioerodible, polymeric device for subcutaneous implantation and sustained release of buprenorphine that has been prewashed with ethanol and exhibits minimal initial burst upon implantation.

### BACKGROUND OF THE INVENTION

Buprenorphine, a semi-synthetic opiate classified as a “partial agonist” behaves very much like classical mu agonists such as morphine, exerting an analgesic effect through high affinity binding to mu subclass opiate receptors in the central nervous system.

Buprenorphine has been used as an analgesic for treatment of moderate to severe pain in postoperative cancer patients. Therapeutic doses administered by intravenous and intramuscular routes range from 0.3 to 0.6 mg. Buprenorphine produces effects similar to morphine but is 25-40 times more potent and has a large therapeutic index. Buprenorphine stimulates the mu opiate receptors in the brain and produces effects associated with other mu agonists such as morphine. Such effects include analgesia, euphoria, sedation, and respiratory depression. For this reason, oral buprenorphine formulations have the potential for misuse (i.e., diversion for recreational, rather than therapeutic, purposes), making them unsuitable for use as a take-home medication.

Many patients with chronic pain require long-term continuous dosing with opiate analgesics. Effective treatment often necessitates the ingestion of multiple tablets per day. Compliance with this dosing scheme is often poor. Further, enteral drug delivery is poorly tolerated or prohibited in patients with particular indications, such as some patients with cancer-related pain in whom continuous drug delivery is a necessity. However, continuous parenteral delivery of opiate analgesics is expensive, cumbersome, and dependent upon the availability of refrigeration, catheters, pumps, and trained personnel. Further, concerns over drug diversion for illicit use often limits availability of opiate analgesics. An oral tablet containing a combination of buprenorphine and naloxone, an opiate receptor antagonist, has been developed to address this issue (see, e.g., U.S. Pat. No. 4,935,428). If an opiate addict attempts to abuse the combination tablet by dissolving it and injecting it intravenously, unpleasant withdrawal symptoms brought on by the naloxone component will result. However, opiate antagonists such as naloxone may reduce the analgesic effectiveness of buprenorphine to those who are using it therapeutically for pain relief and such antagonists may cause undesirable side effects. Thus, there is a need for an improved buprenorphine formulation which is administrable to those in need of analgesia but which provides a lower potential for abuse.

There is a need for a buprenorphine formulation, suitable for long-term, continuous administration, that will improve compliance for pain relief regimens. Subcutaneously implantable polymeric devices have been described for long-term administration of buprenorphine. (U.S. Patent Application No. 2004/0033250) An initial burst of drug is often released upon implantation of a subcutaneously implantable device. Depending upon the magnitude of the initial burst, a large initial release of buprenorphine can cause adverse side effects such as nausea, dizziness, and vomiting. Thus, there is a need for an implantable buprenorphine-containing formulation in which the initial burst of buprenorphine released upon implantation is minimized.

### BRIEF SUMMARY OF THE INVENTION

The invention provides compositions (i.e., implantable polymeric devices), methods, and kits for treatment of pain or a condition for which administration of buprenorphine with low initial burst would be therapeutically beneficial.

In one aspect, the invention provides a subcutaneously implantable device for treatment of pain or a condition for which administration of buprenorphine with a low initial burst would be therapeutically beneficial, comprising buprenorphine and a biocompatible, nonerodible polymer, wherein said buprenorphine is blended with said polymer, and wherein when said implantable device is subcutaneously implanted in a mammal, said buprenorphine is continuously released in vivo over a sustained period of time through pores that open to the surface of said matrix with a low initial burst. In some embodiments, the device does not comprise a coating that is impermeable to buprenorphine. In some embodiments, the device does not comprise external medical equipment. Devices of the invention are prewashed with ethanol to achieve a low initial burst and a desired ratio of peak to average steady state release, such as about 1.2 to about 3.0, about 2.2 to about 2.8, or about 2.4 to about 2.6. The devices may be prewashed with ethanol for any of about 45, 60, 125, 150, 175, 200, 225, 250, 275, 300, 250, 400, 450, 500, 550, 600, 650, 700, 720, 750, or 800 minutes, and/or for a sufficient amount of time to achieve a low initial burst and a low peak to steady state plasma level ratio. Washing with ethanol removes surface buprenorphine and reduces the initial burst of buprenorphine released in vivo after subcutaneous implantation relative to an unwashed device. One or a multiplicity of devices may be administered to release buprenorphine at a therapeutically effective steady state rate of at least about 0.1 mg per day, generally in the range of about 0.1 to about 15 mg per day, In some embodiments, the steady state rate of buprenorphine release is any of about 0.05, 0.1 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, or 14.5 mg per day. In some embodiments, the polymeric matrix comprises EVA. In some embodiments wherein the implantable device comprises EVA, the vinyl acetate content is about 28 to about 40%, for example, about 33%, by weight. The implantable devices generally comprise about 10% to about 67%, often about 50% to about 67% buprenorphine. In one embodiment, the implantable device comprises about 58% buprenorphine and about 42% EVA. In another embodiment, the implantable device comprises about 65% buprenorphine and about 35% EVA. In various embodiments, the sustained period of time for buprenorphine release is from about 3 months to about 1 year, or longer, e.g., at least about 3, 6, 9, or 12 months. In some embodiments, the implantable device is produced by an extrusion process, followed by washing in ethanol to produce a device with a low initial burst, for example, with a peak to steady state plasma level ratio as described herein. In one embodiment, extruded devices comprise dimensions of about 2.4 mm in diameter and about 2.6 cm in length. In other embodiments, extruded devices comprise dimensions of about 2 to about 3 mm in diameter and about 2 to about 3 cm in length. In further embodiments, extruded devices comprises dimensions of about 0.5 to about 7 mm in diameter and about 0.5 to about 10 cm in length.

In another aspect, the invention provides a delivery system for treatment of pain or another condition for which administration of buprenorphine with a low initial burst would be therapeutically beneficial, comprising a multiplicity of implantable devices as described above, wherein when said multiplicity of implantable devices is subcutaneously implanted in a mammal, the combination of devices continuously releases buprenorphine at steady state rate of at least about 0.1 mg per day.

In another aspect, the invention provides a method for treatment of pain, comprising subcutaneously administering at least one implantable device as described above to an individual in need of treatment of pain, wherein the device exhibits a low initial burst after implantation, for example, with a peak to steady state plasma level ratio as described herein, wherein said at least one device continuously releases buprenorphine at an analgesically effective steady state rate for treatment of pain, wherein each of said at least one implantable devices comprises buprenorphine blended with a biocompatible, nonerodible polymer. In some embodiments, one or a multiplicity of devices is administered that is capable of releasing a total of at least any of about 0.05, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 mg buprenorphine per day in vivo. In some embodiments, the devices release a total amount of buprenorphine that results in an average steady state plasma level of any of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 ng/ml. In one embodiment, the method comprises implanting a delivery system comprising a multiplicity of devices, wherein when said multiplicity of implantable devices is subcutaneously implanted in an individual in need of treatment for pain, the combination of implantable devices releases buprenorphine at an analgesically effective steady state rate for treatment of pain in the individual. In various embodiments, one or a multiplicity of devices is implanted for analgesically effective buprenorphine release over a sustained period of time, for example, from about 3 months to about 1 year, or longer, e.g., at least about 3, 6, 9, or 12 months. In some embodiments, implantable devices for treatment of pain are produced by an extrusion process, followed by washing with ethanol for greater than 30 minutes. In methods of the invention, the implantable devices are administered by subcutaneous implantation. In various embodiments, the devices are subcutaneously implanted at a site selected from the group consisting of the upper arm, the back, and the abdomen.

In another aspect, the invention provides a kit for use in treatment of pain, comprising at least one implantable device, as described herein, in packaging, optionally with instructions for use in a method as described herein for treatment of pain or another condition for which administration of buprenorphine with a low initial burst would be therapeutically beneficial.

In another aspect, the invention comprises a process for producing a subcutaneously implantable device comprising buprenorphine and a biocompatible, nonerodible polymeric matrix, comprising washing the device in ethanol for a time period sufficient to produce a device with a low initial burst and a low peak to steady state buprenorphine plasma level ratio, for example, about 1.2 to about 3.0, about 2.2 to about 2.8, or about 2.4 to about 2.6. In one embodiment, the process comprises extruding buprenorphine and a biocompatible, nonerodible polymer, for example, EVA, to produce an implantable device, followed by washing the device with ethanol for any of about 45, 60, 125, 150, 175, 200, 225, 250, 275, 300, 350, 400, 450, 500, 550, 600, 650, 700, 720, 750, or 800 minutes.

### BRIEF DESCRIPTION OF THE DRAWINGS

FIG. 1 depicts buprenorphine plasma levels in dogs over 8 days after subcutaneous implantation of eight polymeric devices containing EVA and buprenorphine. NPPPP1c(WA60): ethanol prewash 250 minutes, buprenorphine content 60 mg per device. NPPPP1d(WA70): ethanol prewash 125 minutes, buprenorphine content 70 mg per device. PRO-510-05-01(80): ethanol prewash 30 minutes, buprenorphine content 80 mg per device.

FIG. 2 depicts buprenorphine data in dogs over 98 days after subcutaneous implantation of eight polymeric devices containing EVA and buprenorphine. This data is from the same experiment as depicted in FIG. 1, but represents a longer time frame after implantation.

### DETAILED DESCRIPTION OF THE INVENTION

The invention provides a biocompatible, nonerodible polymeric device, which permits controlled, sustained release of buprenorphine over extended periods of time when implanted subcutaneously in an individual in need of treatment with buprenorphine, wherein the device has been prewashed with ethanol for an amount of time that results in a low initial burst upon subcutaneous implantation. In some embodiments, the device releases buprenorphine with a peak to steady state plasma level ratio of about 1.2 to about 3, about 2.2 to about 2.8, or about 2.4 to about 2.6. In one embodiment, the peak to steady state ratio may is measured by measuring the peak level at about 3 hours after implantation and the steady state average over about 21 to about 90 days post-implantation. The invention also provides a biocompatible, nonerodible polymeric device, which permits controlled, sustained release of buprenorphine over extended periods of time when implanted subcutaneously in an individual in need of treatment with buprenorphine, wherein the device has the in vivo release characteristics (i.e., after subcutaneous implantation) of a device that has been prewashed with ethanol as described herein to achieve minimal initial burst and release kinetics such as a peak to steady state plasma level ratio as described herein. The invention also includes methods of using the devices of the invention for treatment of pain or another condition for which long term, continuous administration of buprenorphine with a low initial burst would be therapeutically beneficial, and kits comprising one or more implantable devices as described herein.

Continuous release of a compound in vivo over an extended duration may be achieved via implantation of a device containing the compound encapsulated in a nonerodible polymeric matrix. Examples of implantable, nonerodible polymeric devices for continuous drug release are described in, e.g., U.S. Pat. Nos. 4,883,666, 5,114,719, and 5,601,835. Implantable devices improve compliance with drug dosing regimens, due to continuous release of the drug from the polymeric matrix, and reduce abuse potential since such devices are not as subject to diversion as other formulations, such as, for example, oral dosage forms.

Implantation of the device and extended release of buprenorphine limits or prevents diversion of the drug to nonintended users and improves compliance with dosing regimens, eliminating the need for repeated injections or ingestion of pills or tablets. An implantable, sustained-release device according to the present invention also permits achievement of more constant blood levels of buprenorphine than injectable or oral dosage forms, thereby minimizing side effects.

Previous vehicles for long term delivery of buprenorphine have included biodegradable polymers (U.S. Pat. No. 5,486,362). However, in contrast to the present invention, such polymers do not release compounds at linear rates for extended time periods of several months or longer, because channels form in the matrix as it erodes, resulting in increased release rates over time. The present invention includes a biocompatible, nonerodible polymer that exhibits generally linear release kinetics for buprenorphine in vivo, after an initial burst.

Buprenorphine-containing Implantable Polymeric Devices

The invention provides implantable devices for treatment of pain, or any other condition for which continuous, long term administration of buprenorphine with a low initial release rate would be therapeutically beneficial. Devices of the invention include buprenorphine encapsulated in a polymeric, nonerodible matrix and exhibit minimal initial burst when implanted subcutaneously. A low initial burst is advantageous for treatment of pain, because it may reduce or eliminate adverse side effects associated with administration of buprenorphine.

As used herein, “buprenorphine” refers to buprenorphine free base and pharmaceutically acceptable salts thereof, such as buprenorphine HCl. Incorporation of buprenorphine into the polymeric matrix causes the formation of a series of interconnecting channels and pores that are accessible to the surface for release of the drug. The drug is released from the device by diffusion after subcutaneous implantation. Where appropriate, a coating that is impermeable to the drug is placed over at least a portion of the device to further regulate the rate of release. When implanted subcutaneously, devices of the invention continuously release buprenorphine for an extended period of time with a pseudo or near zero order release rate. After an initial burst following implantation, release rates are typically within about 10-20% of the steady state average.

Devices of the invention have the advantageous feature of low initial burst of buprenorphine release in vivo after implantation. In one embodiment, preparation of the implantable device comprises prewashing with ethanol for an amount of time that will result a low initial burst in relation to the average steady state release rate, such as, for example, a ratio of peak to steady state average of about 1.2 to about 3.0, about 2.2 to about 2.8, or about 2.4 to about 2.6, wherein the peak is measured at about 3 hours after implantation and the steady state level is the average plasma level over about 21 to about 90 days post-implantation. In various embodiments, the device is prewashed for any of about 45, 60, 125, 150, 175, 200, 225, 250, 275, 300, 350, 400, 450, 500, 550, 600, 650, 700, 720, 750, or 800 minutes. In some embodiments, the device is prewashed for any of about 45, 60, 125, 150, 175, 200, 225, 250, 275, 300, 350, 400, 450, 500, 550, 600, 650, 700, 720, or 750 minutes, with an upper limit of any of about 60, 125, 150, 175, 200, 225, 250, 275, 300, 350, 400, 450, 500, 550, 600, 650, 700, 720, 750, or 800 minutes. In one embodiment, the invention provides a subcutaneously implantable device comprising buprenorphine and a nonbioerodible polymeric matrix, wherein the device has equivalent in vivo release characteristics as a device that has been washed for any of the ethanol wash times as described above and/or that results in a peak to steady state plasma level ratio as described above.

Prewashing with ethanol generally removes surface buprenorphine and may also remove some subsurface buprenorphine as well, depending on the wash time. Prewashing with ethanol has the added advantage of permitting reduction of overall drug content of the device by adjusting the wash time to remove a desired amount of buprenorphine from the device, to achieve a desired final concentration and total drug content of buprenorphine in the polymeric matrix. For example, as described in Example 1, washing a device containing 75% buprenorphine (w/w in EVA) in 95% ethanol for 125 minutes resulted in a device containing 65% buprenorphine. Washing the same device in 95% ethanol for 250 minutes resulted in a device containing 58% buprenorphine.

Initial burst is typically reduced significantly with respect to an unwashed device or a device washed in ethanol for 30 minutes or less. For example, as discussed in Example 2 below, a buprenorphine-EVA device prewashed for 30 minutes with ethanol and containing 80 mg of buprenorphine exhibited a peak to steady state plasma level ratio of 3.5 after subcutaneous implantation in dogs. In contrast, a device washed for 125 minutes and containing 70 mg of buprenorphine exhibited a peak to steady state ratio of 2.6, and a device washed for 250 minutes and containing 60 mg of buprenorphine exhibited a ratio of 2.4.

In some embodiments, devices of the invention exhibit a peak to steady state plasma level ratio of any of about 1.2 to about 3.0, about 2.0 to about 3.0, about 2.2 to about 2.8, or about 2.4 to about 2.6, after subcutaneous implantation in a mammal. In various embodiments, subcutaneous implantation of a device as described herein results in a peak to steady state plasma level ratio of any of about 1.2, 1.4, 1.6, 1.8, 2.0, 2.2, 2.4, 2.6, 2.8, or 3.0, wherein the peak is measured about 3 hours after subcutaneous implantation and the steady state level is the average from about day 21 to about day 90 post-implantation. In some embodiments, the ratio is any of at least about 1.2, 1.4, 1.6, 1.8, 2.0, 2.2, 2.4, or 2.6, with an upper limit of any of about 1.4, 1.6, 1.8, 2.0, 2.2, 2.4, 2.6, 2.8, or 3.0.

Applicants have found that prewashing of implants having the same dimensions for different lengths of time in the same volume of ethanol does not substantially effect the steady state plasma levels obtained after subcutaneous implantation, although the total drug content and initial burst are reduced as the amount of ethanol wash time is increased. Although not wishing to be bound by theory, this may be due to removal of surface and subsurface buprenorphine, which slows down the initial release of buprenorphine from the polymeric matrix, but buprenorphine is released at a constant rate at steady state due to diffusion through pores that open at the surface of the implant, and the surface area determines the release rate rather than the total drug content in the device.

In one embodiment, the invention provides a subcutaneously implantable device comprising buprenorphine and a nonbioerodible polymeric matrix, wherein the device has equivalent in vivo release characteristics as a device that has been washed with ethanol for greater than 30 minutes, for example, for any of the wash times as described above, for example, exhibiting a peak to steady state plasma level ratio in vivo as described above.

As used herein, “nonerodible matrix” refers to a polymeric carrier that is sufficiently resistant to chemical and/or physical destruction by the environment of use such that the matrix remains essentially intact throughout the release period. The polymer is generally hydrophobic so that it retains its integrity for a suitable period of time when placed in an aqueous environment, such as the body of a mammal, and stable enough to be stored for an extended period before use. The ideal polymer must also be strong, yet flexible enough so that it does not crumble or fragment during use. Nonerodible matrices remain intact in vivo for extended periods of time, typically months or years. Drug molecules encapsulated in the matrix are released over time via diffusion through channels and pores in a sustained and predictable manner. The long term release rate can be altered by modifying the percent drug loading, porosity of the matrix, structure of the implantable device, or hydrophobicity of the matrix, or by adding a hydrophobic coating to at least a portion of the exterior of the implantable device.

Typically, ethylene vinyl acetate copolymer (EVA) is used as the polymeric matrix, but other nonerodible materials may be used. Examples of other suitable materials include silicone, hydrogels such as crosslinked poly(vinyl alcohol) and poly(hydroxy ethylmethacrylate), acyl substituted cellulose acetates and alkyl derivatives thereof, partially and completely hydrolyzed alkylene-vinyl acetate copolymers, unplasticized polyvinyl chloride, crosslinked homo- and copolymers of polyvinyl acetate, crosslinked polyesters of acrylic acid and/or methacrylic acid, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, and chlorosulphonated polyolefines, and combinations thereof.

Implantable devices of the invention are typically formulated with buprenorphine loading of about 10% to about 67% (w/w) after washing. Often, the devices include about 58% to about 65% buprenorphine, and about 52% or 35% EVA, respectively (33% vinyl acetate). In various embodiments, devices include any of at least about 10, 20, 30, 40, 50, 55, 58, 60, or 65% buprenorphine, with an upper limit of any of about 20, 30, 40, 50, 55, 58, 60, 65, or 67% buprenorphine.

In some embodiments, devices of the invention include about 40 to about 75 mg buprenorphine. In some embodiments, the devices include any of about 40, 45, 50, 55, 60, 65, 70, or 75 mg buprenorphine. In some embodiments, the devices include any of at least about 40, 45, 50, 55, 60, 65, 70 mg buprenorphine, with an upper limit of any of about 45, 50, 55, 60, 65, 70, or 75 mg buprenorphine.

Devices may be produced using an extrusion process, wherein ground EVA is blended with buprenorphine, melted, and extruded into rod-shaped structures. Rods are cut into individual implantable devices of the desired length, packaged, and sterilized prior to use. In one embodiment, the devices are prewashed with ethanol as described herein after extrusion and before cutting of the extruded material into individual implants. In another embodiment, the devices are prewashed with ethanol as described herein after extrusion and after cutting of the extruded material into individual implants. Other methods for encapsulating therapeutic compounds in implantable polymeric, nonerodible matrices are well known to those of skill in the art. Such methods include, for example, solvent casting (see, e.g., U.S. Pat. Nos. 4,883,666, 5,114,719, and 5,601,835) and molding. A skilled artisan would be able to readily determine an appropriate method of preparing such an implantable device, depending on the shape, size, drug loading, and release kinetics desired for a particular type of patient or clinical indication.

Devices of the invention are suitable for sustained release of buprenorphine for treatment of pain. As used herein, “sustained release” refers to the release of buprenorphine such that the blood concentration remains within the therapeutic range but below toxic levels for an extended duration. Devices of the invention generally exhibit near zero-order pharmacokinetics in vivo, similar to kinetics achieved with an IV drip, but without the need for external medical equipment and personnel associated with intravenous methods. Generally, after implantation, the devices release therapeutically effective amounts of buprenorphine for periods of several months up to one year or longer. Often, the duration of implantation, with continuous release of buprenorphine, is from about 3 months to about 6 months, about 6 months to about 9 months, about 9 months to about 1 year, or about 1 year to about 2 years. In various embodiments, therapeutically effective amounts of buprenorphine are released for at least about 3, 6, 9, 12, 15, 18, 21, or 24 months. In some embodiments, therapeutically effective amounts of buprenorphine are released for any of about 3, 6, 9, 12, 15, 18, or 21 months, with an upper limit of any of about 6, 9, 12, 15, 18, 21, or 24 months.

Multiple implantable devices may be used, or the size and shape of the devices may be modified, to achieve a desired overall dosage. In some embodiments, the invention provides a delivery system comprising a multiplicity of individual implantable devices as described herein, wherein the multiplicity (i.e., the combination of the individual devices) releases a therapeutically effective amount of buprenorphine in vivo after implantation of the multiplicity of devices in an individual.

Implantable devices of the invention are often about 0.5 to about 10, more often about 1.5 to about 5, most often about 2 to about 3 cm in length, and are often about 0.5 to about 7, more often about 1.5 to about 5, most often about 2 to about 3 mm in diameter. The implantable devices may be any of about 0.5, 1, 1.5, 2, 2.5, 3, 3.5, 4, 4.5, 5, 5.5, 6, 6.5, 7, 7.5, 8, 8.5, 9, or 9.5 cm with an upper limit of any of about 1, 1.5, 2, 2.5, 3, 3.5, 4, 4.5, 5, 5.5, 6, 6.5, 7, 7.5, 8, 8.5, 9, 9.5, or 10 cm in length, and any of about 0.5, 1, 1.5, 2, 2.5, 3, 3.5, 4, 4.5, 5, 5.5, 6, or 6.5 with an upper limit of any of about 1, 1.5, 2, 2.5, 3, 3.5, 4, 4.5, 5, 5.5, 6, 6.5, or 7 mm in diameter.

The release rate of the implantable devices may also be modified by changing the vinyl acetate content in the EVA polymer matrix. The vinyl acetate content is often about 2 to about 40, more often about 10 to about 35, most often about 30 to about 35% by weight. The implantable devices may contain any of about 2, 5, 10, 15, 20, 25, 30, or 35% with an upper limit of any of about 5, 10, 15, 20, 25, 30, 35, or 40% vinyl acetate in the EVA polymer matrix.

The desired dosage rate will depend upon factors such as the underlying condition for which buprenorphine is being administered, and the physiology of a particular patient, but will be readily ascertainable to physicians. As used herein, “therapeutically effective amount” or “therapeutically effective level” refers to the amount of buprenorphine that will render a desired therapeutic outcome (e.g., analgesic relief of pain). For treatment of pain, a therapeutically effective amount of buprenorphine is typically about 0.1 to about 15 mg/day, sometimes about 0.2 to about 1 mg/day, often about 0.3 mg/day, but may be modified depending upon the nature of the pain condition being treated and the particular patient involved. In various embodiments for treatment of pain, one or a multiplicity of devices is used that is capable of releasing a total of at least any of about 0.05, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, or 14.5 mg/day with an upper limit of any of about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 mg/day in vivo or in vitro. In various embodiments for treatment of pain, one or a multiplicity of devices is used that is capable of releasing a total amount of buprenorphine in vivo that will result in an average steady state plasma level of about 0.1 to about 2 ng/ml.

In some embodiments, the devices release a total amount of buprenorphine that results in an average steady state plasma level of any of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 ng/ml. In some embodiments, the devices release a total amount of buprenorphine that results in an average steady state plasma level of any of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, or 1.9 ng/ml, with an upper limit of any of about the devices release a total amount of buprenorphine that results in an average steady state plasma level of any of about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 ng/ml.

Methods of Preparation

The invention provides a method for preparation of a biocompatible, nonerodible polymeric device, which permits controlled, sustained release of buprenorphine over extended periods of time when implanted subcutaneously in an individual in need of treatment with buprenorphine, wherein implantation of a device with a low initial burst is advantageous, such as treatment of pain. The method comprises prewashing the device with ethanol prior to subcutaneous implantation in an individual to produce a device with a desired peak to steady state plasma level ratio, with a low initial burst, such as, for example, a ratio of about 1.2 to about 3.0, about 2.2 to about 2.8, or about 2.4 to about 2.6. In one embodiment, the peak is measured at about 3 hours after subcutaneous implantation and the steady state is the average over a period of about 21 to about 90 days post-implantation. In one embodiment, 95% ethanol is used for prewashing of the device. In some embodiments, the device is prewashed for any of about 45, 60, 125, 150, 175, 200, 225, 250, 275, 300, 350, 400, 450, 500, 550, 600, 650, 700, 720, 750, or 800 minutes. In some embodiments, the device is prewashed for any of about 45, 60, 125, 150, 175, 200, 225, 250, 275, 300, 350, 400, 450, 500, 550, 600, 650, 700, 720, or 750 minutes, with an upper limit of any of about 60, 125, 150, 175, 200, 225, 250, 275, 300, 350, 400, 450, 500, 550, 600, 650, 700, 720, 750, or 800 minutes.

In some embodiments, the device is prewashed with ethanol until a desired overall buprenorphine concentration and total drug content in the device is achieved, such as, for example, about 40 to about 75 mg buprenorphine. In some embodiments, the devices include any of about 40, 45, 50, 55, 60, 65, 70, or 75 mg buprenorphine. In some embodiments, the devices include any of at least about 40, 45, 50, 55, 60, 65, 70 mg buprenorphine, with an upper limit of any of about 45, 50, 55, 60, 65, 70, or 75 mg buprenorphine. In some embodiments, the buprenorphine concentration in the device is about 10% to about 67% (w/w) after washing. Often, the devices include about 58% to about 65% buprenorphine, and about 42% or 35% EVA, respectively. In various embodiments, devices include any of at least about 10, 20, 30, 40, 50, 55, 58, 60, or 65, % buprenorphine, with an upper limit of any of about 20, 30, 40, 50, 55, 58, 60, 65, or 67% buprenorphine.

In some embodiments, the ethanol wash volume is varied as well as the wash time to achieve an implant with a desired buprenorphine concentration or total drug loading. For example, an implant having dimensions of about 2.4 mm in diameter and about 2.6 cm in length may be washed in a volume of any of about 2, 5, 10, 20, 30, 40, 50, 60, 70, 80, 90, or 100 ml of ethanol at any of the wash times described above, to achieve a desired buprenorphine concentration or total drug content.

In some embodiments, the device is prewashed with ethanol until a device having desired in vivo release characteristics after subcutaneous implantation is produced (i.e., with a low initial burst and/or a peak to steady state plasma level ratio as described above).

Methods of Treatment

The invention provides methods for treatment of pain. The invention also provides methods for treatment of any indication for which continuous, long term administration of buprenorphine with a low initial burst would be therapeutically beneficial. Administration of buprenorphine via devices of the invention with low or minimal initial burst are particularly advantageous for patient populations that have not been habitual opiate users, for example, patients who need treatment for pain and who are not opiate abusers or who do not have a history of opiate administration at high levels. Side effects from an initial burst of buprenorphine in patients who have not previously been administered opiates could be harmful, and the devices of the invention solve this problem by minimizing the initial burst of buprenorphine to which these patients are exposed.

Methods of the invention include subcutaneous administration of one or more polymeric implantable devices which contain buprenorphine encapsulated within a biocompatible, nonerodible polymeric matrix such as EVA, and release of a therapeutically effective amount of buprenorphine in a controlled manner over an extended period of time through multiple pores that open to the surface of the implantable device(s). Often, implantable devices are produced via an extrusion process, as described above.

In methods of the invention, implantation of the device(s) results in minimal initial burst. As discussed above, a device of the invention has been prewashed in ethanol as described herein or has the release characteristics (e.g., initial burst and steady state release kinetics) of a device that has been prewashed in ethanol as described herein. In methods of the invention, the lower initial burst may have the advantage of reducing or eliminating uncomfortable or adverse side effects associated with a large initial release of buprenorphine, such as nausea, dizziness, and vomiting. In some embodiments, the peak to steady state plasma level ratio after subcutaneous implantation of one or more devices as described herein is any of about 1.2 to about 3.0, about 2.0 to about 3.0, about 2.2 to about 2.8, or about 2.4 to about 2.6. In one embodiment, the peak is measured at about 3 hours after subcutaneous implantation and the steady state is the average from about 21 to about 90 days post-implantation. In various embodiments, subcutaneous implantation of a device as described herein results in a peak to steady state plasma level ratio of any of about 1.2, 1.4, 1.6, 1.8, 2.0, 2.2, 2.4, 2.6, 2.8, or 3.0. In some embodiments, the ratio is any of at least about 1.2, 1.4, 1.6, 1.8, 2.0, 2.2, 2.4, or 2.6, with an upper limit of any of about 1.4, 1.6, 1.8, 2.0, 2.2, 2.4, 2.6, 2.8, or 3.0.

Implantable devices are administered by subcutaneous implantation to an individual in need of treatment. As used herein, “individual” refers to a mammal, such as a human, in need of treatment for pain. Generally, implantable devices are administered by subcutaneous implantation at sites on the upper arm, back, or abdomen of an individual. Other suitable sites for administration may be readily determined by a medical professional. A delivery system comprising multiple implantable devices may be administered to achieve a desired dosage for treatment.

In accordance with the present invention, a method of alleviating pain is provided which includes subcutaneous administration of an implantable nonerodible polymeric device as described herein which releases buprenorphine in a controlled manner for an extended period of time, i.e. about 3 months to about 1 year (e.g., at least about 3, 6, 9, or 12 months) or longer, to an individual exhibiting pain, with a low initial burst upon implantation. In some embodiments, the device releases buprenorphine with a peak to steady state plasma level ratio of about 1.2 to about 3.0, about 2.2 to about 2.8, or about 2.4 to about 2.6. In one embodiment, the peak is measured at about 3 hours after subcutaneous implantation and the steady state average is determined over the time period from about 21 to about 90 days post-implantation.

One or a multiplicity of devices is administered to achieve a total buprenorphine release rate that results in a therapeutically effective buprenorphine plasma level for analgesic relief of pain. An implantable device or multiplicity of devices is chosen for a particular patient or pain indication such that a therapeutically effective amount of buprenorphine will be continuously released from the device or devices. In one embodiment, a single device is administered that continuously releases buprenorphine at a rate that results in a therapeutically effective plasma level. In another embodiment, a delivery system is administered that comprises a multiplicity of devices (e.g., 2, 3, 4, or more devices) wherein the combination of devices continuously releases buprenorphine at a rate that results in a therapeutically effective plasma level. In various embodiments for treatment of pain, one or a multiplicity of devices is used that is capable of releasing a total amount of buprenorphine in vivo that will result in an average steady state plasma level of about 0.1 to about 2 ng/ml. In some embodiments, the devices release a total amount of buprenorphine that results in an average steady state plasma level of any of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 ng/ml. In some embodiments, the devices release a total amount of buprenorphine that results in an average steady state plasma level of any of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, or 1.9 ng/ml, with an upper limit of any of about the devices release a total amount of buprenorphine that results in an average steady state plasma level of any of about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 ng/ml.

As used herein, the term “analgesically effective amount” refers to an amount of buprenorphine sufficient to achieve a reduction in or elimination of pain in a mammalian subject without loss of consciousness. The effectiveness of analgesia provided by an analgesic substance can be assessed by methods that are well known to those skilled in the art. For example, U.S. Pat. No. 6,228,863 describes assessment by direct measurement in human patients or by the use of one or more surrogate measures. Direct measurement may include scoring an analgesic questionnaire reported by patients at serial times following administration of the analgesic substance. Summary measures of analgesia include the sum of pain intensity difference (SPID) and total pain relief (TOTPAR). Surrogate measures for determining analgesic effectiveness in human subjects include assessment of sedation, respiratory rate and/or pupil size, and visual analogue scale for drug effect. Effectiveness of analgesia may also be assessed using animal models. For example, U.S. Pat. No. 4,599,342 describes a mouse hot plate test and a phenylquinone writhing test model for assessing the extent of analgesia.

The method of alleviating pain according to the present invention is applicable to the treatment of all pain conditions which require continuous administration of an analgesic substance, e.g., postoperative pain, cancer pain, arthritic pain, lumbosacral pain, musculoskeletal pain, neuropathic pain, rheumatic pain, neuralgia, etc. This list, however, should not be interpreted as exhaustive. It is contemplated that the method of the invention will improve compliance with dosing regimens for therapeutic pain relief, while reducing the amount of opiate substances that are available for diversion to nonintended users, i.e., drug addicts. Long term continuous release should also reduce or eliminate the peaks and troughs of blood analgesic concentration associated with other formulations such as oral dosage forms. Typically, the steady state release rate of buprenorphine used for pain relief methods is from about 0.1 to about 15, sometimes about 0.2 to about 1, often about 0.3 mg per day, but may be modified depending upon the nature of the pain condition being treated and the particular patient involved. In various embodiments for treatment of pain, one or a multiplicity of devices is used that is capable of releasing a total of at least any of about 0.05, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, or 14.5 mg/day with an upper limit of any of about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 mg/day in vivo.

In any of the above methods, the length of time during which buprenorphine is continuously administered may be extended by reimplanting additional implantable devices in an individual receiving treatment before or after plasma levels of buprenorphine begin to decline, to maintain buprenorphine at the desired steady state level.

Kits

The invention also provides kits for use in treatment of pain or another condition for which continuous, long term administration of buprenorphine with a minimal initial burst is therapeutically beneficial. The kits include at least one implantable, nonerodible device of the type herein described, capable of delivering long-term therapeutic levels of buprenorphine with minimal initial burst, in suitable packaging, along with instructions providing information to the user and/or health care provider regarding subcutaneous implantation and use of the system for treating pain or another condition for which continuous, long term administration of buprenorphine with a minimal initial burst is therapeutically beneficial. Kits may also include literature discussing performance of the implantable devices of the invention. Kits may include a delivery system, i.e., a multiplicity of implantable devices, capable of providing sustained release of therapeutic levels of buprenorphine for at least about 3 months.

Kits for treatment of pain typically contain a delivery system capable of releasing a total continuous analgesically effective dosage of about 0.1 to about 15, about 0.2 to about 1, or about 0.3 mg buprenorphine per day for at least about 3 months. In various embodiments for treatment of pain, a kit comprises one or a multiplicity of devices that is capable of releasing a total of at least any of about 0.05, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, or 14.5 mg/day with an upper limit of any of about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 mg/day in vivo or in vitro. In kits of the invention, implantable devices may be preloaded into devices such as, for example, syringes or trocars, capable of administering them by subcutaneous implantation into patients.

The following examples are intended to illustrate but not limit the invention.

### EXAMPLES

### Example 1

### Preparation of Buprenorphine-Containing Polymeric Implants

Polymeric implants were prepared by twin screw hot melt extrusion. The extrusion process involved subjecting an EVA and buprenorphine HCL blend (75% buprenorphine/25% EVA) to pressure and heat with a Thermo-Prism Twin Screw Extruder. The extruder melted the EVA and formed it into long thin segments of solid material (the extrudate). The extrudate was cut manually into segments of approximately 15 (+/−3) inches in length as the material was extruded. (The lengths were arbitrarily chosen based on ease of handling). The segments were then cut into implants with a predetermined length specification of 26 mm.

The implants were washed in 190 proof ethanol USP for different lengths of time remove surface buprenorphine and to reduce the drug load to a desired level. Implants with 75% buprenorphine were washed with ethanol for 250 minutes to produce implants containing 60 mg of buprenorphine (58% w/w) or for 125 minutes to produce implants containing 70 mg of buprenorphine (65% w/w).

The 60 mg implants were prepared as follows: 150 implants (75% (w/w) buprenorphine, 135.9 mg total weight) were placed in a clean stainless steel screen and submerged in 2438.7 grams Alcohol USP. The alcohol was agitated for 250 minutes at 115 to 270 rpm using a 3 inch Teflon coated magnetic stir bar. The temperature of the alcohol was 21.5° C. The alcohol was tested using UV-Vis spectroscopy to determine Buprenorphine content. An average of 39.0 mg of Buprenorphine HCl was removed from each implant. The remaining drug load in each implant was therefore approximately 60 mg. The implants were air dried for 20 minutes. The implants were then placed into an oven equipped with a vacuum set at 30° C. and 27 inches Hg for 12.5 hours.

The 70 mg implants were prepared as follows: 150 implants (75% (w/w) buprenorphine, 137.0 mg total weight) were placed in a clean stainless steel screen and submerged in 2439.6 grams Alcohol USP. The alcohol was agitated for 125 minutes at 115 to 270 rpm using a 3 inch Teflon coated magnetic stir bar. The temperature of the alcohol was 21.6° C. The alcohol was tested using UV-Vis spectroscopy to determine Buprenorphine content. An average of 30.3 mg of Buprenorphine HCl was removed from each implant. The remaining drug load in each implant was therefore approximately 70 mg. The implants were air dried for 65 minutes. The implants were then placed into an oven equipped with a vacuum set at 30° C. and 27 inches Hg for 12.5 hours.

### Example 2

### In vivo Release Characteristics of Buprenorphine-Containing Implants

In vivo buprenorphine release characteristics of polymeric implants prepared as described in Example 1 were assessed by subcutaneous implantation in dogs. Twenty adult male beagle dogs were used for the study. Implants containing 60 mg buprenorphine (250 min ethanol wash) were subcutaneously implanted in eight dogs, implants containing 70 mg buprenorphine (125 min ethanol wash) were subcutaneously implanted in eight dogs, and implants containing 80 mg buprenorphine (30 min ethanol wash) were subcutaneously implanted in four dogs. Blood samples were collected prior to implantation and then 3, 6, 9, and 12 hours, and 1, 2, 3, 4, 5, 6, 7, 14, 28, 35, 42, 49, 56, 63, 70, 84, and 98 days after implantation, to determine buprenorphine plasma levels.

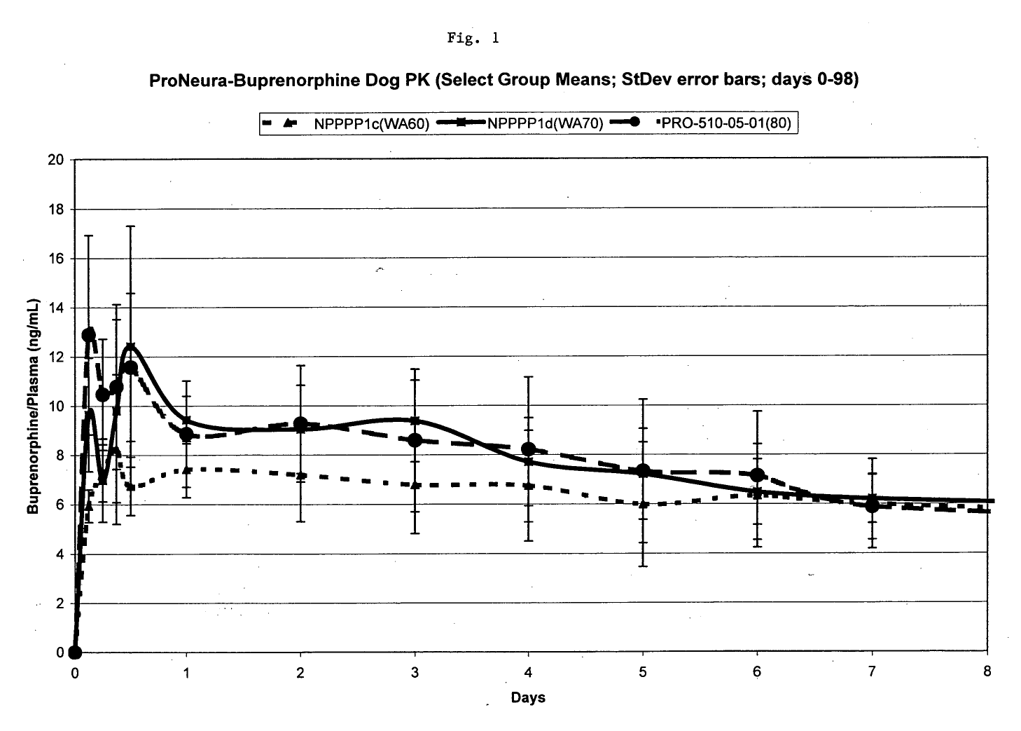

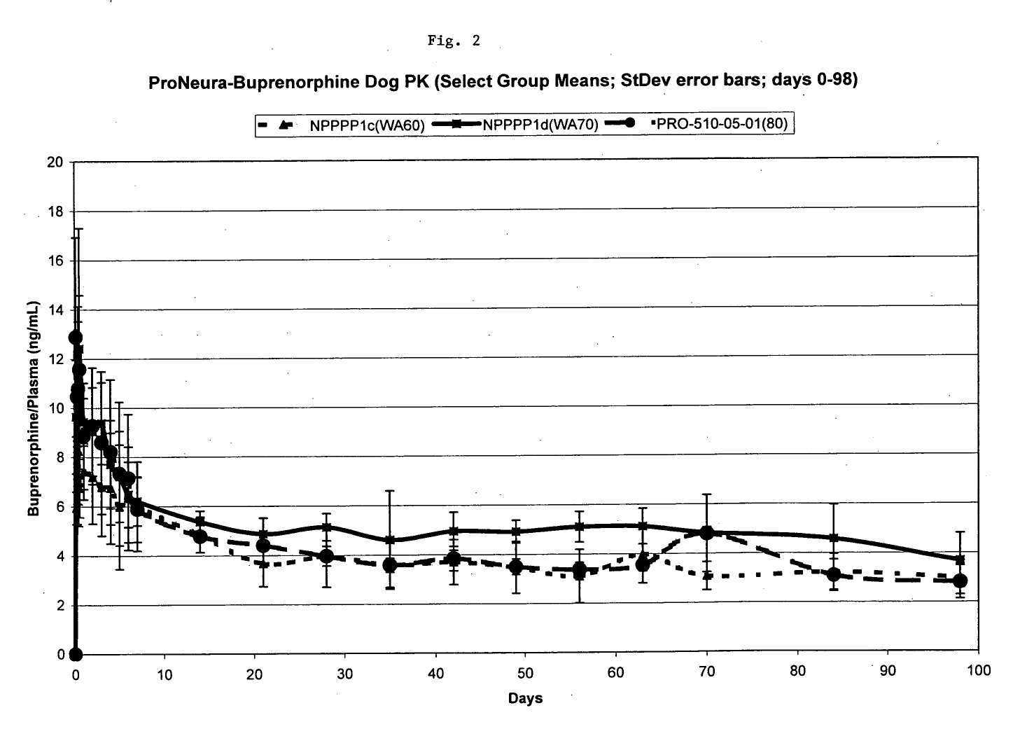

The results are shown in FIGS. 1 and 2. FIG. 1 shows the buprenorphine release profile over the first 8 days post-implantation. A low initial burst was observed with the 60 mg implants (NPPPP1c(WA60)), a slightly higher initial burst is observed with the 70 mg implants (NPPPP1d(WA70)), and a higher initial burst was observed with the 80 mg implants (PRO-510-05-01(80)). FIG. 2 shows the buprenorphine release profile over 98 days, during which a steady state of buprenorphine release is achieved. The steady state release was observed to be relatively constant for the three devices, and independent of the amount of buprenorphine loading in the device. The ratio of peak (3 hours) to steady state release (21 to 90 days) was 2.4 for the 60 mg implants, 2.6 for the 70 mg implants, and 3.5 for the 80 mg implants.

All publications, patents, and patent applications cited herein are hereby incorporated by reference in their entireties for all purposes and to the same extent as if each individual publication, patent, or patent application were specifically and individually indicated to be so incorporated by reference.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

We claim:

1. An implantable device comprising buprenorphine and a biocompatible, nonerodible polymer, wherein said device comprises a polymeric matrix comprising buprenorphine blended with said polymer, and wherein when said implantable device is implanted subcutaneously in a mammal, said buprenorphine is continuously released in vivo over a sustained period of time through pores that open to the surface of said matrix, wherein said device is washed with ethanol prior to subcutaneous implantation, wherein said washing with ethanol removes surface buprenorphine and reduces the initial burst of buprenorphine released in vivo after subcutaneous implantation relative to an unwashed device, and wherein the ratio of peak to average steady state release of buprenorphine is about 1.2 to about 3.

2. An implantable device according to claim 1, wherein the device is washed with ethanol for about 125 minutes.

3. An implantable device according to claim 1, wherein the device is washed with ethanol for about 250 minutes.

4. An implantable device according to claim 1, wherein the polymeric matrix comprises ethylene vinyl acetate copolymer (EVA).

5. An implantable device according to claim 4, wherein said EVA comprises about 33% vinyl acetate.

6. An implantable device according to claim 5, comprising about 10 to about 67% buprenorphine.

7. An implantable device according to claim 4, wherein the implantable device is produced by an extrusion process.

8. An implantable device according to claim 7, comprising dimensions of about 0.5 to about 7 mm in diameter and about 0.5 to about 10 cm in length.

9. An implantable device according to claim 8, comprising dimensions of about 2.4 mm in diameter and about 2.6 cm in length.

10. An implantable device according to claim 9, wherein when said implantable device is implanted subcutaneously in a mammal, buprenorphine is continuously released from the device at a steady state release rate of at least about 0.1 mg buprenorphine per day.

11. An implantable device according to claim 9, wherein when said implantable device is implanted subcutaneously in a mammal, buprenorphine is continuously released from the device at a rate that results in a steady state plasma buprenorphine level of at least about 0.1 ng/ml

12. An implantable device according to claim 1, wherein the sustained period of time is at least about 3 months.

13. A method for treating pain in a mammal in need thereof, comprising administering at least one implantable device according to claim 1 subcutaneously to said mammal, wherein said device continuously releases an analgesically effective amount of buprenorphine for treatment of pain for a sustained period of time.

14. A method according to claim 13, wherein the device is washed with ethanol for about 125 minutes.

15. A method according to claim 13, wherein the device is washed with ethanol for about 250 minutes.

16. A method according to claim 13, wherein the polymeric matrix comprises ethylene vinyl acetate copolymer (EVA).

17. A method according to claim 16, wherein said EVA comprises about 33% vinyl acetate.

18. A method according to claim 17, comprising about 10 to about 67% buprenorphine.

19. A method according to claim 16, wherein the implantable device is produced by an extrusion process.

20. A method according to claim 19, comprising dimensions of about 0.5 to about 7 mm in diameter and about 0.5 to about 10 cm in length.

21. A method according to claim 20, comprising dimensions of about 2.4 mm in diameter and about 2.6 cm in length.

22. A method according to claim 13, wherein when said implantable device is implanted subcutaneously in said mammal, buprenorphine is continuously released from the device at a steady state release rate of at least about 0.1 mg buprenorphine per day.

23. A method according to claim 13, wherein when said implantable device is implanted subcutaneously in said mammal, buprenorphine is continuously released from the device at a rate that results in a steady state plasma buprenorphine level of at least about 0.1 ng/ml

24. A method according to claim 13, wherein the sustained period of time is at least about 3 months.

25. A method according to claim 13, wherein said at least one implantable device comprises a multiplicity of individual implantable devices, and wherein the combination of said implantable devices continuously releases buprenorphine in vivo over a sustained period of time at an analgesically effective steady rate for treatment of pain in said mammal.

26. A method according to claim 13, wherein each of said at least one implantable devices is subcutaneously implanted at a site selected from the group consisting of the upper arm, the back, and the abdomen.

27. A kit for use in treatment of pain, comprising at least one implantable device according to claim 1.

28. A process for producing a subcutaneously implantable device comprising buprenorphine and a biocompatible, nonerodible polymeric matrix, said process comprising washing said device in ethanol, wherein the initial burst of buprenorphine released in vivo after subcutaneous implantation is reduced relative to an unwashed device, wherein the ratio of peak to average steady state release of buprenorphine is about 1.2 to about 3.

29. A process according to claim 28, wherein said device is washed in ethanol for about 125 minutes.

30. A process according to claim 28, wherein said device is washed in ethanol for about 250 minutes.

31. A delivery system for treatment of pain, comprising a multiplicity of implantable devices according to claim 1, wherein when said multiplicity of devices is implanted subcutaneously in a mammal, buprenorphine is continuously released in vivo at a rate that results in an analgesically effective steady state rate for treatment of pain.

32. A kit for use in treatment of pain, comprising a delivery system according to claim 31.
